(19) United States
(12) Patent Application Publication
(10) Pub. No.: US 2026/0354682 A1
(43) Pub. Date: Nov. 12, 2026

(54) Apparatus for Eye Examination

(71) Applicant: EYOTO GROUP LIMITED, Birmingham (GB)

(72) Inventors: Alex Bamford, Birmingham (GB); Deepkamal Braich, Birmingham (GB); Joseph Davies, Birmingham (GB)

(21) Appl. No.: 19/134,336

(22) PCT Filed: Dec. 7, 2023
(86) PCT No.: PCT/GB2023/053158

§ 371 Date: May 30, 2025

(51) Int. Cl. A61B 3/12; A61B 3/14

(52) U.S. Cl. CPC A61B 3/12; A61B 3/14

(57) ABSTRACT

An eye examination apparatus 1 comprises a movable slit lamp 10 and a movable camera 20. A fundus lens 60 is aligned with the camera 20 by mounting assembly 100 comprising a plinth 110 and a bracket 120. The plinth 110 comprises a pair of notches 119 defined between side fingers 117, 118, each notch 119 having a relatively wide opening and narrowing towards a blind end. The bracket 120 comprises a pair of projecting lugs 129. The narrowing profile of the notches 119 guides a corresponding lug 129 as the bracket 120 is brought together with the plinth 110 until the lugs 129 abut the curved blind end of the notches 119 when the bracket 120 and plinth are in the desired alignment. A magnet 130 on plinth 110 holds the bracket 120 in position for use.

### TECHNICAL FIELD OF THE INVENTION

The present invention relates to apparatus for eye examination and in particular to apparatus for eye examination using a slit lamp. More particularly, the invention relates to a mounting assembly for a lens for such apparatus.

### BACKGROUND TO THE INVENTION

In many instances, it is necessary or useful to examine the eye. One way in which such examination is carried out is by use of slit lamp operable to produce an intense, narrow beam of light. By suitable direction of this beam into and across the eye of a patient, examination of various structures of the eye can be carried out. This examination can be carried out by directly viewing the illuminated eye or by viewing images of the illuminated eye captured using a suitable digital camera.

In many instances a dedicated apparatus is provided to aid in performing such examinations. The apparatus may comprise a chin rest upon which the patient can rest their chin so as correctly locate their eye relative to the apparatus and a slit lamp, provided on an illumination arm movable relative to the apparatus. Movement of the illumination arm can provide control of the direction and location of illumination by the slit lamp. The apparatus can further be provided with optics, or a camera provided with a suitable lens assembly and positioned so as to capture images of the eye whilst under illumination by the slit lamp. The optics, or camera may be mounted on a viewing arm movable relative to the apparatus so as to control the position of the optics or camera relative to the eye under examination.

Traditionally, an operator has positioned the patient on the chin rest and manually manipulated the apparatus to capture images. The operator may be an ophthalmologist who examines the images directly or may be a technician who stores the captured images for subsequent examination by an ophthalmologist. More recent apparatus provides for motor control of the illumination arm and/or viewing arm. This can be carried out by a local operator using a control interface provided on the apparatus or by a remotely located operator sending control signals from a suitable control interface. The remote operator may also receive a stream of captured images from the camera in order to provide feedback on operation as well as images for examination.

Whilst such a set up provides for many common eye examination requirements, detailed examination of the posterior surface (fundus) of the eye requires additional equipment over and above the standard features discussed above. Such detailed examination typically requires the use of an additional fundus lens which relays the beam produced by the slit lamp to the fundus while simultaneously relaying an image of the fundus to the camera. Given the competing requirements on the fundus lens of magnification and field of view, the fundus lens may be scanned across the eye or positioned so as to allow detailed examination of particular features. Traditionally, this has been achieved by manipulation of a fundus lens held by an operator. Typically, the fundus lens would be provided with an annular housing adapted to be gripped by the operator. Such manipulation requires a reasonable level of skill and training and also requires the operator to be in close contact with the patient.

It is proposed that the required movement to achieve examination can be emulated by means of a fundus lens mounted such that it is accurately aligned with the lens assembly of a movable camera. Whilst such an arrangement would not be expected to match the flexibility of skilled manual manipulation of a fundus lens, it would be possible to control such movement via a local or remote control interface. In order for such an arrangement to be implemented the fundus lens must be mounted sufficiently securely that it does not move relative to the camera and the mounting arrangement must be sufficiently lightweight so as not to detract from camera movement. In order to facilitate examinations both with and without the fundus lens, the mounting arrangement must facilitate removal of the fundus lens from alignment with the camera and accurate replacement. No existing mounting arrangement is known that would adequately provide for the above requirements.

It is therefore an object of the present invention to at least partially overcome or alleviate some of these issues.

### SUMMARY OF THE INVENTION

According to a first aspect of the present invention there is provided a mounting assembly for a lens, the mounting assembly comprising a bracket and a plinth, the bracket comprising an arm extending between a tip and a base wherein the tip comprises a lens holder, and wherein the base and plinth comprise co-operating features which interact when the base is in a desired alignment with the plinth and wherein the base or the plinth comprises a magnet.

The co-operating features of the mounting arrangement of the present invention thus facilitate simple alignment of the bracket and plinth. The use of a magnet provides for the bracket and plinth to be readily and securely retained in position once alignment is achieved and for the bracket to be readily removed when required. The mounting arrangement is also relatively lightweight and thus does not significantly impede motor control if fitted to a camera.

The plinth and bracket may be formed from metal. In other embodiments, one of the plinth and the bracket may be formed from metal and the other may be formed from a plastic material. In such embodiments the one of the base or plinth comprising a magnet may be formed from a plastic material and the other of the plinth or base may be formed from a magnetic material. In such embodiments, the metal may be a magnetic metal, and in some embodiments a ferromagnetic metal. Suitable plastic materials include but are not limited to nylon or the like.

The co-operating features may comprise a plurality of projecting lugs and a corresponding series of notches. In one embodiment, the lugs are provided on the plinth and the notches are provided on the base of the bracket. In an alternative embodiment, the notches are provided on the plinth and the lugs are provided on the base of the bracket.

In a particular arrangement there are two lugs and two notches. In other arrangements, there may be more lugs and notches.

The notches may have a profile comprising a relatively wide opening narrowing towards a blind end. In some embodiments, this may be a V profile. In the desired alignment, the lugs may abut the blind end of the notch profile. The narrowing profile of the notches thus helps with alignment of the mounting assembly. The blind end of the narrowing profile may comprise a single vertex, multiple vertices or a curved wall.

The lugs may have any suitable transverse cross-section. In some embodiments, the transverse cross-section of the lugs may be elongate. In other embodiments, the transverse cross-section of the lugs may be substantially regular. The lugs may have a curved transverse cross-section. The curved transverse cross-section may be substantially circular or substantially oval. The curved transverse cross-section may comprise a wedge having a curved nose for abutting the blind end of the notches.

The blind end of the notches may be curved or otherwise adapted to match the transverse cross-section of the lugs.

Each notch may have a substantially similar profile. In alternative embodiments, each notch may have a different profile. In such embodiments, each lug may have a different profile corresponding to only one of the different notch profiles. This can further ensure correct alignment and may be useful in arrangements where the co-operating features could be fitted together in multiple configurations.

Between and/or to either side of the notches, there may be one or more fingers. In some embodiments, any finger between two notches may extend further than fingers at only one side of the notches. This helps with alignment of the mounting assembly.

The magnet may be a permanent magnet. The magnet may be held in position by suitable magnet fixing means. The magnet fixing means may comprise snap-fitting elements, screws, pins, bolts, adhesive or the like.

In some embodiments, the bracket may be provided with the magnet and the plinth may be formed from magnetically susceptible material. In other embodiments, the plinth may be provided with the magnet and the bracket may be formed from magnetically susceptible material. In further embodiments, the bracket may be provided with one magnet and the plinth may be provided with an opposingly orientated magnet.

In some embodiments, the magnet may be fitted within a recess. The recess may be provided in the plinth or bracket as appropriate. The recess may correspond in size and profile to the magnet such that the magnet surface is in a common plane with the surface surrounding the recess. The recess may be positioned such that it fully accepts the magnet when the bracket and plinth are in the desired alignment. In other embodiments, the magnet may project proud of the surface to which it is fitted.

The plinth may be provided with fixing means for securely fitting the plinth to an article. In many embodiments, the article is an optical article to which a lens is to be removably fitted at a desired separation. The optical article may be a camera or the like. The plinth fixing means may comprise may include snap-fitting elements, screws, pins, bolts, adhesive or the like.

The bracket arm may extend along a single linear axis. Alternatively, the bracket arm may comprise two linear sections meeting at an elbow. In further embodiments, the bracket arm may be curved. The bracket arm may have a substantially constant cross-sectional profile. In other embodiments, the cross-sectional profile of the bracket arm may be varied.

In some embodiments, the lens holder may be releasably fitted to the tip. In other embodiments, the lens holder may be integrally formed with the tip.

The lens holder may be adapted to fit any suitable form or lens. In a particular embodiment, the lens holder may be adapted to hold a fundus lens.

According to a second aspect of the present invention there is provided an eye examination apparatus comprising a slit lamp provided on an illumination arm movable relative to the apparatus; camera mounted on a camera arm movable relative to the apparatus; and a mounting assembly for a lens according to the first aspect of the present invention, wherein the plinth is fitted to the camera.

The apparatus of the second aspect of the present invention may incorporate any or all features of the first aspect of the present invention, as desired or as appropriate.

The slit lamp may be provided within a protective housing. The protective housing may comprise an aperture and a light guiding arrangement operable to direct light emitted from the slit lamp in a desired direction relative to the housing. The light guiding arrangement may comprise one or more mirrors, lenses or the like.

The camera may be provided within a protective housing. The housing may be adapted to enable the fixing of the plinth of the mounting assembly. The adaptations may include co-operating features for the fixing means of the plinth. For instance, where the fixing means are screws, the co-operating features may be threaded screw holes. The camera housing may also comprise a lensing arrangement. The lensing arrangement may be adapted to focus incident light on the sensing element of the camera.

The apparatus may comprise a chin rest. The chin rest may be positioned such that the patient can rest their chin so as correctly locate their eye relative to the apparatus. The chin rest may be adjustable. The chin rest may be mounted to a supporting frame.

Movement of the illumination arm may be implemented by one or more motors. Similarly, movement of the camera arm may be implemented by one or more motors. In some embodiments, the illumination arm and the camera arm may be rotatable about a common axis.

The or each motor may be controlled by a control unit. The control unit may be operable in response to a operator interface. The operator interface may be a local operator interface or may be a remote operator interface in communication with the control unit. In suitable embodiments, communication can be implemented over any suitable communication link. Such communication links may be wireless, wired or a combination thereof.

The operator interface may comprise operator actuated input means. The operator actuated input means may comprise any one or more of the following: push buttons, key pads, touch sensitive areas, joysticks, rotary switches, toggle switches, pointing devices or the like. The operator interface may be provided with a display unit.

In suitable embodiments, images captured by the camera may also be communicated to the local operator interface or remote operator interface. The communicated captured images may be output via the local or remote operator interface. This can facilitate for remote examination of captured images. It can also facilitate a remote operator controlling the examination process.

The apparatus may comprise a microphone and/or a loudspeaker. The remote operator interface may comprise a microphone and/or a loudspeaker. In such embodiments, the communication link may be operable to transmit audio signals between the apparatus and operator interface. This can enable communication between a remote operator and a patient.

According to a third aspect of the present invention there is provided a method of aligning a fundus lens with a camera of an eye examination apparatus, the method comprising the steps of providing a mounting assembly according to the present invention; fitting the plinth of the mounting assembly to a housing of the camera; bringing the bracket base into contact with the plinth; and aligning the co-operating features of the base of the bracket with the co-operating features of the plinth base of the bracket so as to secure the bracket in position relative to the plinth.

The method of the third aspect of the present invention can incorporate any or all features of the first or second aspects of the present invention as desired or as appropriate.

The method may include the further step of removing the backet from the plinth, when desired.

### DETAILED DESCRIPTION OF THE INVENTION

In order that the invention may be more clearly understood one or more embodiments thereof will now be described, by way of example only, with reference to the accompanying drawings, of which:

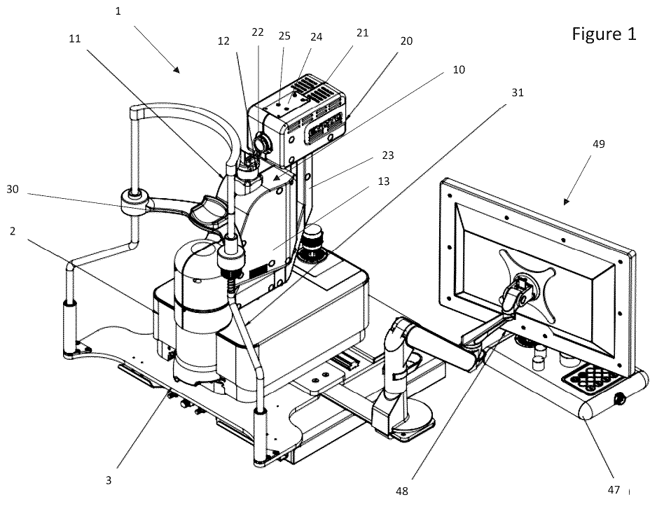

FIG. 1 shows an eye examination apparatus for use with the mounting assembly of the present invention;

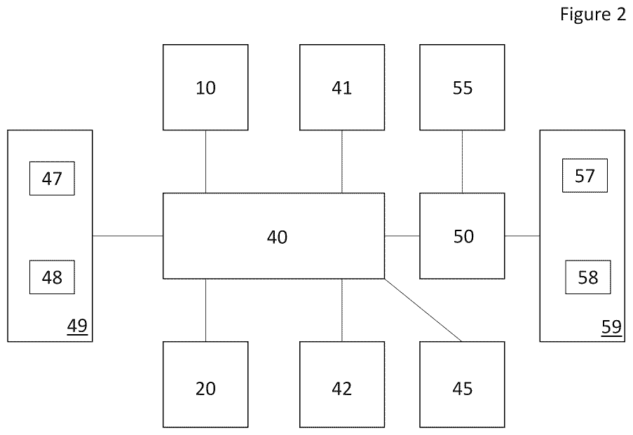

FIG. 2 is a schematic block diagrams of the operative elements of the eye examination apparatus of FIG. 1.

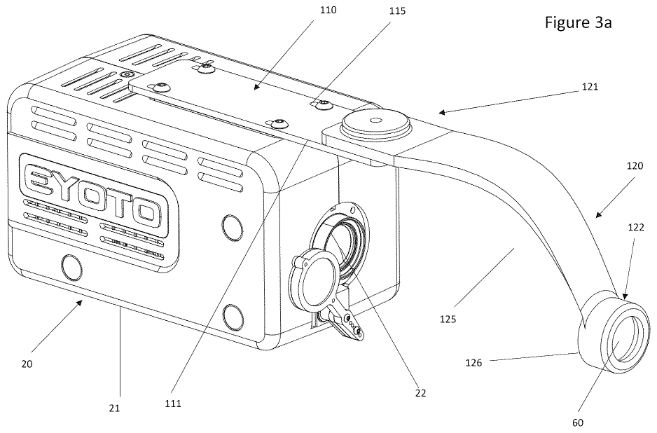

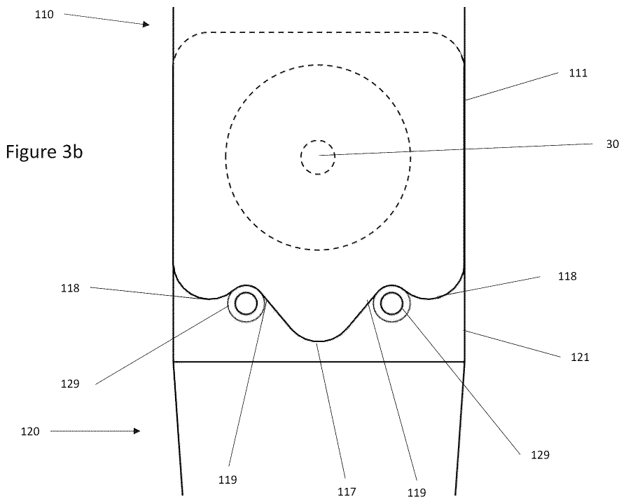

FIG. 3a shows a camera for use in the eye examination apparatus of FIG. 1, provided with a mounting assembly for a lens according to the present invention;

FIG. 3b shows an expanded view illustrating the interaction between a plinth and a bracket of the mounting assembly in FIG. 3a;

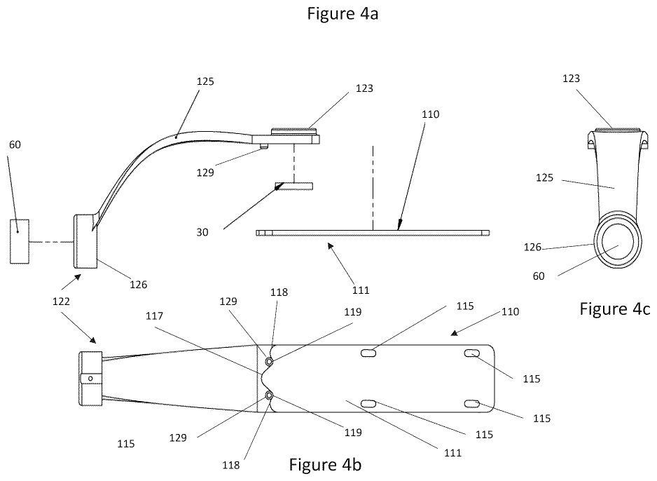

FIG. 4a is an exploded view of the mounting assembly of the present invention;

FIG. 4b is a bottom view of the mounting assembly of the present invention; and

FIG. 4c is an end view of the mounting assembly of the present invention.

Turing to FIG. 1, an eye examination apparatus 1 comprises a slit lamp 10 provided within a protective housing 11. The slit lamp 10 is provided with a light guiding arrangement 12 operable to direct light emitted from the slit lamp 10 in a desired direction relative to the housing. The slit lamp 10 is provided on an illumination arm 13 movable relative to the apparatus 1.

The eye examination apparatus 1 also comprises a camera 20 provided within a protective housing 21. The camera 20 is provided with a lensing arrangement 22 adapted to focus incident light on the sensing element of the camera 20. The camera is provided on a camera arm 23 movable relative to the apparatus 1. The camera 20 is further adapted to enable the fixing of a plinth 110 of a mounting assembly 100 according to the present invention, as described further below. In this example, the adaptation comprises a mounting area 24, which may comprise a reinforced plate in some embodiments. Within the mounting are located one or more mounting holes 25. Typically, these holes 25 are adapted to retain fixing elements such as screws, for instance by being provided with an interior thread.

The eye examination apparatus 1 also comprises a chin rest 30, enabling a patient to rest their chin so as correctly locate their eye relative to the apparatus 1. The chin rest 30 may be adjustably mounted on a support frame 31.

The eye examination apparatus 1 shown in FIG. 1 also comprises main body 2, having a support base 3. Typically, within the main body is provided a control unit 40. Additionally, the main body 2 may also typically comprise a power source or connection to a power source. The illumination arm 13 and camera arm 23 are both rotatable about a common axis 4, provided on main body 2. Motors, 41, 42 (see FIG. 2) for driving the illumination arm 13 and camera arm 23 respectively may be provided in the main body 2.

The eye examination apparatus 1 is provided with a operator interface 49 comprising a display unit 48 and operator actuable inputs 47. As shown in the example, the operator actuable inputs 47 include a mixture of push buttons and rotary controls. The skilled person will appreciate that any suitable combination of the operator actuable inputs 47 can be provided, as is required or as is convenient for the operator. In some embodiment, the operator interface may additionally be provided with a microphone, camera and/or loudspeaker (none shown).

Turing now to FIG. 2, a schematic block diagram of the apparatus 1 is shown. The operator interface 49 is connected to the control unit 40. This allows the control unit 40 to output information relating to the operation of the apparatus 1 to display 48. Similarly, this allows the control unit 40 to operate in response to operator actuation of operator actuable inputs 47.

The camera 20 and slit lamp 10 are activated in response to control signals generated by the control unit 40. Similarly, the illumination arm motor 41 and camera arm motor 42 are activated in response to the control unit 40. These control signals may be dependent on the operator actuation of operator actuable inputs 47. In some cases, the control signals may be directly translated from the operator actuation. In other cases, the control signals may be a pre-set routine initiated in response to the operator actuation.

Images captured by the camera 20 are output to the control unit 40. Accordingly, they can subsequently be output via display unit 48 for inspection by an operator. Output camera images can also be stored for future reference in a local data store 45.

As shown in FIG. 2, the apparatus may additionally comprise a communication unit 50. The communication unit 50 allows the control unit to transmit and receive data signals over a suitable wired or wireless data connection. In such embodiments, output camera images and other operational data may be transmitted to an external data store 55 for later retrieval by the present apparatus or other devices. Additionally or alternatively, the apparatus 1 may be connected to a remotely located operator interface 59 comprising at least a display unit 58 and operator actuable inputs 57. This can enable operation of the apparatus 1 to be controlled by a remotely located operator. In such embodiments, the apparatus 1 may be provided with a loudspeaker and microphone. This can facilitate communication between a remote operator and the patient. Additionally, in such embodiments, an additional wide angle camera may be provided, which may enable the remote operator to better understand the position of the patient, in cases where alignment proves difficult.

In use, a patient rests their chin on the suitably adjusted chin rest 30. The control unit is subsequently operable to generate control signals to activate the slit lamp 10 and camera 20. Additionally, the control unit 10 is operable in response to operator inputs received from operator actuable inputs 47 (or operator actuable inputs 57) to generate control signals to for the illumination arm motor 41 and camera arm motor 42. The control signals move the illumination arm 13 and camera arm 23 and hence move slit lamp 10 and camera 20 relative to the patient's eye so as to capture images of the patient's eye under illumination. These captured images can then be output on the local display 48 (or remote display 58) for examination by an operator.

In order to facilitate detailed examination of the posterior surface (fundus) of the eye, use of an additional fundus lens 60 is required. The fundus lens 60 sits between the patient's eye and the slit lamp 10 and camera 20. As such, the fundus lens relays the beam produced by the slit lamp 10 to the fundus while simultaneously relaying an image of the fundus to the camera 20. Traditionally, this has been achieved by manipulation of a fundus lens 60 held by an operator. In the present invention, the fundus lens 60 is instead mounted such that it is accurately and securely aligned with the lens assembly 22 of the camera 20 using a mounting assembly 100 according to the present invention as illustrated in FIGS. 3 and 4.

The mounting assembly 100 allows the fundus lens 60 can be readily fitted when fundus examination is required and readily removed when fundus examination is not required. Furthermore, the mounting assembly 100 is sufficiently lightweight that is does not adversely affect movement of camera 20 but mounts the fundus lens 60 sufficiently securely that it does not move relative to the camera 20.

The mounting assembly 100 comprises a plinth 110 and a bracket 120. In the example shown, the plinth 110 is in the form of a plate fitted to the camera housing 21, with an overhanging end 111. As illustrated, the plinth 110 can be fitted to the mounting area 24 by use of screws (not shown) which screw into screw holes 25 through holes 115. The bracket 120 comprises an arm 125 extending between a tip 122 and a base 121 wherein the tip comprises a lens holder 126 for retaining fundus lens 60. The base 121 of the bracket 120 and plinth 110 comprise co-operating features which interact when the base 121 is in a desired alignment with the plinth 110.

The overhanging end 111 of plinth 110 comprises a pair of notches 119 defined between side fingers 118 and central finger 117. The notches 119 have a profile with relatively wide opening narrowing towards a blind end, the blind end being curved in this example. The skilled person will of course appreciate that the blind end could have a sharp vertex or form other than a curve in different embodiments.. The base 121 of bracket 120 is provided with a pair of projecting lugs 129. The narrowing profile of the notches 119 helps guide a corresponding lug 129 towards the blind end as the bracket 120 is brought together with the plinth 110. When the bracket 120 is in the desired alignment with the plinth 110, the lugs 129 abut the curved blind end of the notches 119. As illustrated, the curve of each blind end substantially matches the transverse cross-section of each lug 129. This ensures that the lug 129 can closely abut the blind end of notch 119. This in turn provides for reliable and accurate alignment of the bracket 120 with the plinth 110 and hence of the fundus lens 60 with the lensing assembly 22 of the camera 20, with simple manipulation of the bracket 120 relative to the plinth 110. The skilled person will appreciate that in alternative arrangements, the lugs 129 could be provided on the plinth 110 and the notches 119 could be provided on bracket 120.

To secure the retention of the alignment between the plinth 110 and bracket 120, a magnet 130 is provided. The magnetic attraction between the magnet 130 and the plinth 110 holds the bracket 120 in position sufficiently securely for use in fundus examination. Nevertheless, an operator can readily remove the bracket 120 from the plinth 110 by overcoming the magnetic attraction. To maximise the security of the magnetic attraction in the example embodiment, the plinth 110 can be formed from a ferromagnetic material.

In the example shown, the magnet 130 is fitted into a recess 123 provided in the base 121 of bracket 120. The magnet 130 can be retained in the recess 123 by any suitable fixing means, for example adhesive. The recess 123 enables the surface of magnet 130, which in this example is in the form of a disk, to lie in a common plane with the surface of the bracket base 121.

Whilst the magnet 130 in the example is fitted to the bracket 120, the skilled person will appreciate that the magnet 130 could alternatively be fitted to the plinth 110. Additionally or alternatively, the skilled person will appreciate that opposingly orientated magnets 130 could be provided on each of the plinth 110 and bracket 120.

The one or more embodiments are described above by way of example only. Many variations are possible without departing from the scope of protection afforded by the appended claims.

1. A mounting assembly for a lens, the mounting assembly comprising a bracket and a plinth, the bracket comprising an arm extending between a tip and a base wherein the tip comprises a lens holder, and wherein the base and plinth comprise co-operating features which interact when the base is in a desired alignment with the plinth and wherein the base or the plinth comprises a magnet,

2. A mounting assembly as claimed in claim 1 wherein the co-operating features comprise a plurality of projecting lugs and a corresponding series of notches.

3. A mounting assembly as claimed in claim 2 wherein the lugs are provided on the plinth and the notches are provided on the base of the bracket.

4. A mounting assembly as claimed in claim 2 wherein the notches are provided on the plinth and the lugs are provided on the base of the bracket.

5. A mounting assembly as claimed in claim 2 wherein there are two lugs and two notches.

6. A mounting assembly as claimed in claim 2 wherein the notches have a profile comprising a relatively wide opening narrowing towards a blind end.

7. A mounting assembly as claimed in claim 6 wherein the notches have a V profile.

8. A mounting assembly as claimed in claim 6 wherein in the desired alignment, the lugs but the blind end of the notch profile.

9. A mounting assembly as claimed in claim 8 wherein the lugs have a curved transverse cross-section

10. A mounting assembly as claimed in claim 8 wherein the blind end of the notches is curved or otherwise adapted to match the transverse cross-section of the lugs.

11. A mounting assembly as claimed in claim 2 wherein each notch has a substantially similar profile

12. A mounting assembly as claimed in claim 2 wherein between and/or to either side of the notches, there are one or more fingers.

13. A mounting assembly as claimed in claim 12 wherein any finger between two notches extends further than fingers at only one side of the notches.

14. A mounting assembly as claimed in claim 1 wherein the magnet is fitted within a recess corresponding in size and profile to the magnet such that the magnet surface is in a common plane with the surface surrounding the recess.

15. A mounting assembly as claimed in claim 1 wherein the plinth is provided with fixing means for securely fitting the plinth to an article.

16. A mounting assembly as claimed in claim 1 wherein the lens holder is releasably fitted to the tip of the bracket.

17. An eye examination apparatus comprising a slit lamp provided on an illumination arm movable relative to the apparatus; camera mounted on a camera arm movable relative to the apparatus; and a mounting assembly for a lens according to claim 1 wherein the plinth is fitted to the camera.

18. An eye examination apparatus as claimed in claim 17 wherein the slit lamp is provided within a protective housing comprising an aperture and a light guiding arrangement operable to direct light emitted from the slit lamp in a desired direction relative to the housing.

19. An eye examination apparatus as claimed in claim 17 wherein the camera is provided within a protective housing adapted to enable the fixing of the plinth of the mounting assembly and wherein the camera housing comprises a lensing arrangement, adapted to focus incident light on the sensing element of the camera.

20. An eye examination apparatus as claimed in any one of claims 17 wherein movement of the illumination arm and the camera arm is implemented by one or more motors and wherein each motor is controlled by a control unit, operable in response to an operator interface.

21. An eye examination apparatus as claimed in claim 20 wherein the operator interface is a local operator interface or is a remote operator interface in communication with the control unit.

22. An eye examination apparatus as claimed in claim 20 wherein the operator interface comprises operator actuated input means and a display unit and wherein images captured by the camera are communicated to the operator interface for output.

23. A method of aligning a fundus lens with a camera of an eye examination apparatus, the method comprising the steps of providing a mounting assembly according to claim 1; fitting the plinth of the mounting assembly to a housing of the camera; bringing the bracket base into contact with the plinth; and aligning the co-operating features of the base of the bracket with the co-operating features of the plinth base of the bracket so as to secure the bracket in position relative to the plinth.
